(19) United States
(12) Patent Application Publication
(10) Pub. No.: US 2024/0020839 A1
(43) Pub. Date: Jan. 18, 2024

(54) MEDICAL IMAGE PROCESSING DEVICE, MEDICAL IMAGE PROCESSING PROGRAM, AND MEDICAL IMAGE PROCESSING METHOD

(71) Applicant: NIDEK CO., LTD., Gamagori-shi (JP)

(72) Inventors: Ryosuke SHIBA, Gamagori-shi (JP); Yoshiki KUMAGAI, Gamagori-shi (JP)

(21) Appl. No.: 18/477,067

(22) Filed: Sep. 28, 2023

Related U.S. Application Data
(63) Continuation of application No. PCT/JP2022/009329, filed on Mar. 4, 2022.

(30) Foreign Application Priority Data
Mar. 31, 2021 (JP) 2021-059329

(51) Int. Cl. G06T 7/00; G06T 15/00; G06V 10/24; G06V 10/25; G06V 10/75

(52) U.S. Cl. CPC G06T 7/0012; G06T 15/00; G06V 10/24; G06V 10/25; G06V 10/751; G06T 2207/30024

(57) ABSTRACT

A medical image processing device is configured to process data of a three-dimensional image of a biological tissue. The medical image processing device includes a controller configured to: acquire, as an image acquisition step, a three-dimensional image of a tissue; extract, as an extraction step, a first region from the acquired three-dimensional image, the first region being a part of the three-dimensional image; and acquire, as a first structure detection step, a detection result of a specific structure of the tissue in the extracted first region by inputting the first region into a mathematical model that is trained by a machine learning algorithm to output a detection result of a specific structure that is shown in an image input into the mathematical model.

### CROSS REFERENCE TO RELATED APPLICATION

This application is a continuation application of International Patent Application No. PCT/JP2022/009329 filed on Mar. 4, 2022, which designated the U.S. and claims the benefit of priority from Japanese Patent Application No. 2021-059329 filed on Mar. 31, 2021. The entire disclosure of the above application is incorporated herein by reference.

### TECHNICAL FIELD

The present disclosure relates to a medical image processing device that processes image data of biological tissues, a storage medium storing a medical image processing program executed in the medical image processing device, and a medical image processing method.

### BACKGROUND

Traditionally, various techniques have been proposed for detecting a specific structure of a tissue shown in an image (e.g., layers, boundaries of multiple layers, and specific parts within the tissue). For instance, using convolutional neural networks, each pixel is mapped to determine which layer the pixel belongs to. Based on the results of this mapping, boundaries of layers are identified.

Using convolutional neural networks, it is possible to detect a specific structure of a tissue with high accuracy. However, compared to traditional methods using image processing, the computational burden tends to increase. Therefore, when detecting a tissue structure from three-dimensional image data (sometimes referred to as “volume data”), the amount of data to be processed substantially increases. Consequently, it is desirable to reduce processing time. For example, using a GPU, which has higher computational capabilities than a CPU, segmentation of the retinal layers is carried out using the neural network. This approach aims at reducing processing time.

### SUMMARY

The present disclosure provides a medical image processing device configured to process data of a three-dimensional image of a biological tissue. The medical image processing device includes a controller configured to: acquire, as an image acquisition step, a three-dimensional image of a tissue; extract, as an extraction step, a first region from the acquired three-dimensional image, the first region being a part of the three-dimensional image; and acquire, as a first structure detection step, a detection result of a specific structure of the tissue in the extracted first region by inputting the first region into a mathematical model that is trained by a machine learning algorithm to output a detection result of a specific structure that is shown in an image input into the mathematical model.

### BRIEF DESCRIPTION OF DRAWINGS

FIG. 1 is a block diagram showing a schematic configuration of a mathematical model building device, a medical image processing device, and a medical imaging device.

FIG. 2 shows an example of a two-dimensional cross-sectional image of a retina used for training.

FIG. 3 shows an example of output data indicating a specific structure of a tissue depicted in the training image shown in FIG. 2.

FIG. 4 is an explanatory diagram showing a method by the medical imaging device for capturing a three-dimensional image of a living tissue.

FIG. 5 is an explanatory diagram showing a state where a three-dimensional image is formed from multiple two-dimensional images.

FIG. 6 is a flowchart of a first detection process executed by the medical image processing device.

FIG. 7 is an explanatory diagram illustrating a process of classifying multiple A-scan images in a two-dimensional image into multiple groups.

FIG. 8 is a flowchart of a second detection process performed by the medical image processing device.

FIG. 9 is an explanatory diagram showing an example method of extracting a tissue image area of a two-dimensional image based on a reference image.

FIG. 10 is a flowchart of a third detection process executed by the medical image processing device.

FIG. 11 is a diagram comparing two-dimensional images before and after alignment.

FIG. 12 is a flowchart of a fourth detection process performed by the medical image processing device.

FIG. 13 is a reference diagram for explaining the fourth detection process.

FIG. 14 is a flowchart of a fifth detection process executed by the medical image processing device.

FIG. 15 shows an example where an attention point and a extraction pattern are set in a three-dimensional image.

FIG. 16 is a block diagram showing a schematic configuration of a medical image processing system according to a modified example.

### DESCRIPTION OF EMBODIMENTS

Next, a relevant technology will be described first only for understanding the following embodiments. Controllers such as GPUs with high computational capabilities (hereinafter referred to as “high-performance controllers”) cannot be used depending on a situation. Further, high-performance controllers are expensive. Therefore, if computational complexity can be reduced while maintaining high detection accuracy when detecting a tissue structure from a three-dimensional image, it would be highly beneficial.

One of objectives of the present disclosure is to provide a medical image processing device, a storage medium storing a medical image processing program, and a medical image processing method that can reduce computational complexity (computational requirements) while maintaining high detection accuracy when detecting a tissue structure from a three-dimensional image.

In a first aspect of the present disclosure, a medical image processing device is configured to process data of a three-dimensional image of a biological tissue. The medical image processing device includes a controller configured to: acquire, as an image acquisition step, a three-dimensional image of a tissue; extract, as an extraction step, a first region from the acquired three-dimensional image, the first region being a part of the three-dimensional image; and acquire, as a first structure detection step, a detection result of a specific structure of the tissue in the extracted first region by inputting the first region into a mathematical model that is trained by a machine learning algorithm to output a detection result of a specific structure that is shown in an image input into the mathematical model.

In a second aspect of the present disclosure, a non-transitory, computer readable, storage medium stores a medical image processing program for a medical image processing device configured to process data of a three-dimensional image of a biological tissue. The medical image processing program, when executed by a controller of the medical image processing device, causes the controller to perform: acquiring, as an image acquisition step, a three-dimensional image of a tissue; extracting, as an extraction step, a first region from the acquired three-dimensional image, the first region being a part of the three-dimensional image; and acquiring, as a first structure detection step, a detection result of a specific structure of the tissue in the extracted first region by inputting the first region into a mathematical model that is trained by a machine learning algorithm to output a detection result of a specific structure that is shown in an image input into the mathematical model.

In a third aspect of the present disclosure, a medical image processing method is implemented by a medical image processing device configured to process data of a three-dimensional image of a biological tissue. The method includes: acquiring, as an image acquisition step, a three-dimensional image of a tissue; extracting, as an extraction step, a first region from the acquired three-dimensional image, the first region being a part of the three-dimensional image; and acquiring, as a first structure detection step, a detection result of a specific structure of the tissue in the extracted first region by inputting the first region into a mathematical model that is trained by a machine learning algorithm to output a detection result of a specific structure that is shown in an image input into the mathematical model.

According to the medical image processing device, medical image processing program, and medical image processing method of the present disclosure, distortion in images of biological tissues produced by light scanning can be appropriately corrected.

In a typical aspect of the present disclosure, a medical image processing device is configured to process data of a three-dimensional image of a biological tissue. The medical image processing device includes a controller configured to: acquire, as an image acquisition step, a three-dimensional image of a tissue; extract, as an extraction step, a first region from the acquired three-dimensional image, the first region being a part of the three-dimensional image; and acquire, as a first structure detection step, a detection result of a specific structure of the tissue in the extracted first region by inputting the first region into a mathematical model that is trained by a machine learning algorithm to output a detection result of a specific structure that is shown in an image input into the mathematical model.

According to the above-described aspect, a portion of the region is extracted as the first region from the entire three-dimensional image. Detection processing of the specific structure using the mathematical model is executed for the extracted first region. As a result, the computational requirements for processing using a machine learning algorithm can be reduced as compared to applying the mathematical model to the entire three-dimensional image. In the following description, the structure detection process executed by the mathematical model on the first region may be referred to as a “first structure detection process.”

The structure of the tissue to be detected from the image may be chosen as appropriate. For instance, if the image is an ophthalmic image, a target structure may be any of the following or a combination thereof: layers of the subject eye's retinal tissue, boundaries of the retinal tissue layers, optic disc present at the retina, layers of the anterior eye tissue, boundaries of the anterior eye tissue layers, and disease sites of the subject eye.

Furthermore, various devices may be used as an imaging (generation) device for the three-dimensional image. For example, an OCT (Optical Coherence Tomography) device that captures cross-sectional images of tissues using the principle of optical coherence tomography may be used. The imaging methods by OCT devices may be, for instance, scanning a spot of light (measurement light) in two dimensions to obtain a three-dimensional cross-sectional image, or scanning light extending in one dimension to obtain a three-dimensional cross-sectional image (so-called Line-Field OCT). Additionally, MRI (Magnetic Resonance Imaging) devices, CT (Computed Tomography) devices, and the like may also be used.

The control unit may further execute a second structure detection step. In the second structure detection step, the control unit detects a specific structure in the second region, which is part of the entire area of the three-dimensional image but was not extracted as the first region in the extraction step, based on the detection results of the specific structure in the first region that were output from the mathematical model.

In this case, in addition to the structure in the first region, the structure in the second region is also detected. As a result, the specific structure within the three-dimensional image can be detected with higher accuracy. Furthermore, for the detection of the specific structure in the second region, the detection results on for the first region output from the mathematical model are used. Therefore, the computational requirements for the structure detection process on the second region can be less than those for the structure detection process on the first region. Thus, the structure detection processes for both the first and second regions are executed without substantially increasing in computational requirements. In the following description, the structure detection process on the second region based on the detection results of the first structure detection process may be referred to as a “second structure detection process.”

The specific method for executing the second structure detection step (i.e., the specific method for the second structure detection process) may be chosen as appropriate. For example, the control unit may acquire the detection result of the structure in the second region by comparing the detection results and pixel information (e.g., brightness values) of each pixel constituting the first region with the pixel information of each pixel constituting the second region. In this case, the positional relationship between each of pixels constituting the second region and each of pixels constituting the referenced first region (e.g., the first region closest to the target second region) may be taken into account. For instance, the detection results and pixel information of a pixel among the pixels within the first region, which is one of pixels from the closest pixel to a focused pixel in the second region to the n^{th} pixel may be compared with the pixel information of the said focused pixel. Additionally, the control unit may acquire the detection result of the structure as to the focused pixel in the second region by interpolating based on the detection results of the structure as to pixels in the first region surrounding the focused pixel.

The control unit may extract the first region from each of the multiple two-dimensional images constituting the three-dimensional image in the extraction step. In this case, the computational requirements are appropriately reduced as compared to executing the structure detection process by the mathematical model for the entire area of each two-dimensional image.

In the extraction step, the control unit classifies each of multiple rows of pixels constituting the two-dimensional image into one of multiple groups based on the degree of similarity. Then, a row of pixels representing each group may be extracted as the first region. In the first structure detection step, the control unit may input the row of pixels extracted as the first region in the extraction step into the mathematical model. In this situation, even if a large number of rows of pixels are classified into one group, the structure detection process by the mathematical model is executed for one or a few rows of pixels representing the group. Therefore, the computational requirements of the process using the mathematical model can be reduced.

The direction in which the row of pixels extends may be defined as appropriate. For instance, when a three-dimensional image is captured by an OCT (Optical Coherence Tomography) device, among the multiple two-dimensional images that constitute the three-dimensional image, the row of pixels extending in the direction along the optical axis of the OCT light may be referred to an A-scan image. In this case, each of the multiple A-scan images that constitute the two-dimensional image may be classified into one of the multiple groups. Also, each of the multiple rows of pixels that intersect perpendicularly with the A-scan image may be classified into one of the multiple groups.

In addition to the first structure detection process, the above-described second structure detection process may also be executed. In this case, the control unit may detect a specific structure in the row of pixels that was not detected as the first region (i.e., the second region) from each group based on the structure detection results of the mathematical model for the first region of the same group. As mentioned before, the degree of similarity of the multiple rows of pixels classified into the same group is high. Therefore, by executing the first structure detection process and the second structure detection process for each group, the accuracy of the second structure detection process can be further improved.

The specific method for extracting the row of pixels that represents each of the multiple groups as the first region may also be chosen as appropriate. For instance, the control unit may extract the row of pixels obtained by performing an addition-averaging process on the multiple rows of pixels classified into each group as the first region. Moreover, the control unit may extract the first region from the multiple rows of pixels belonging to each group according to a predetermined rule or randomly. In this scenario, the number of the first regions extracted from each group may be one, or it may be multiple, provided that the number is less than the number of rows of pixels belonging to the corresponding group.

However, the method of detecting the structure based on multiple rows of pixels that constitute a two-dimensional image is not necessarily limited to the method of classifying rows of pixels into multiple groups. For instance, the control unit may extract rows of pixels as the first region at regular intervals from the multiple rows of pixels that constitute a two-dimensional image. In this case, the control unit may execute both the process of extracting the first region from multiple rows of pixels aligned in a first direction and the process of extracting the first region from multiple rows of pixels aligned in a second direction perpendicular to the first direction.

A three-dimensional image may be formed by arranging in sequence multiple two-dimensional images in a direction that intersects the tissue image area of each two-dimensional image. In the extraction step, the control unit may extract a rectangular tissue image area where a tissue is depicted as the first region from each of the multiple two-dimensional images. In this case, the area where no tissue is depicted is excluded from the target region from which a specific tissue is detected using a mathematical model. Consequently, the computational load of the processing using the mathematical model is appropriately reduced.

In the extraction step, the control unit may detect the tissue image area of a reference image by inputting a reference image among multiple two-dimensional images into the mathematical model. The control unit may extract the tissue image area of the two-dimensional image other than the reference image as the first region based on the detection results on the reference image. In this case, the tissue image area of the reference image is detected with high accuracy by the mathematical model. Additionally, the tissue image areas of the two-dimensional images other than the reference image are detected with a reduced computational load based on the detection results of the tissue image area of the reference image. Thus, the tissue image areas are detected more appropriately.

It should be noted that the method of extracting the tissue image areas of other two-dimensional images based on the detection results of the tissue image area of the reference image may be chosen as appropriate. For instance, the control unit may extract the tissue image areas of other two-dimensional images by comparing the detection results of the tissue image area for each of pixels constituting the reference image and the pixel information with the pixel information of each of pixels constituting the other two-dimensional images. In this case, the positional relationship between each of the pixels constituting the reference image and each of the pixels constituting the other two-dimensional images may be taken into consideration.

However, the method of extracting the tissue image area from each two-dimensional image may be changed. For example, the control unit may extract the tissue image area based on the pixel information of each of the pixels constituting the two-dimensional image. As one example, the control unit may detect a region where the pixel brightness in the two-dimensional image exceeds a threshold as the tissue image area.

The control unit may further execute a two-dimensional image inter alignment step to align the tissue images between multiple rows of pixels that constitute each two-dimensional image. In the first structure detection step, the rectangular first region, which has been aligned and extracted in the two-dimensional image inter alignment step and the extraction step, may be input into the mathematical model. In this case, by executing both the two-dimensional image inter alignment step and the extraction step, the image fits appropriately within the rectangular first region. Moreover, the size of the rectangular first region tends to decrease. As a result, the structure can be detected appropriately with a reduced computational load.

Note that either the two-dimensional image inter alignment step or the extraction step can be executed first. In other words, after the image alignment was performed between multiple rows of pixels, the rectangular tissue image area may be extracted as the first region. Alternatively, after the tissue image area was identified as the first region, image alignment may be performed between multiple rows of pixels so that the shape of the first region can be adjusted to a rectangular shape.

The control unit may further execute a multiple two-dimensional images alignment step to align the tissue images between multiple two-dimensional images. In this case, the processing is executed more efficiently in various respects. For instance, when detecting a specific structure in one two-dimensional image (i.e., the second region) based on the result of the first structure detection process for another two-dimensional image (i.e., the first region), the control unit aligns the tissue images between the multiple two-dimensional images. In this situation, by comparing pixels with close coordinates between the two-dimensional images, the structure in the second region can be detected more accurately.

Note that either the multiple two-dimensional images alignment step or the extraction step can be executed first. Moreover, either the multiple two-dimensional images alignment step or the two-dimensional image inter alignment step can be executed first.

The control unit, in the extraction step, may extract some of the multiple two-dimensional images contained in the three-dimensional image as the first region. In this case, compared to performing the structure detection process by the mathematical model for all the two-dimensional images constituting the three-dimensional image, the computational load required during the process is appropriately reduced.

The control unit may execute the extraction step and the first structure detection step for the reference image as the first region among the multiple two-dimensional images included in the three-dimensional image. Subsequently, the control unit may execute the extraction step and the first structure detection step for the two-dimensional images, as the first region, among the multiple two-dimensional images that have similarity with the reference image falling below a threshold. The control unit may repeatedly execute the above processes.

For instance, it is possible to extract two-dimensional images at regular intervals as the first region from multiple two-dimensional images constituting the three-dimensional image. However, in this case, even in parts where the structure changes drastically, the first region is extracted only at regular intervals. As a result, there is a possibility that the accuracy of structure detection decreases. On the contrary, by extracting the first region using the degree of similarity with the reference image on which the first structure detection process was executed, the first region is densely extracted in parts where the structure changes drastically. Therefore, the accuracy of structure detection can be improved.

However, the method of extracting the first region on a two-dimensional image basis is not necessarily limited to the method of extracting using the degree of similarity with the reference image. For example, the control unit may extract two-dimensional images at regular intervals as the first region from multiple two-dimensional images that constitute the three-dimensional image.

In the extraction step, the control unit may set an attention point within the tissue image area of the three-dimensional image. The control unit may set an extraction pattern for multiple two-dimensional images based on the set attention point. The control unit may extract multiple two-dimensional images that match the set extraction pattern as the first region from the three-dimensional image. In this case, multiple two-dimensional images are extracted as the first region according to the extraction pattern based on the attention point. Consequently, a specific structure from the three-dimensional image can be detected in an appropriate manner corresponding to the attention site.

The specific method for setting the attention point can be chosen as appropriate. For instance, the control unit can set the attention point within the tissue image area of the three-dimensional image according to instructions input by a user. In this case, the first region is appropriately extracted based on the position the user is focusing on. Moreover, the control unit may detect a specific part in the three-dimensional image (e.g., a part where a specific structure exists or a part where a disease exists, etc.) and may set the detected specific part as the attention point. In this situation, the control unit may use known image processing techniques to detect the specific part. Additionally, a mathematical model may be used to detect the specific part.

The extraction pattern for multiple two-dimensional images can also be chosen as appropriate. For instance, when viewing the three-dimensional image in a direction along the imaging optical axis, the extraction pattern may be set so that lines traversed by the extracted two-dimensional images radially expand from the attention point. Furthermore, the closer it is to the attention point, the extraction pattern may be set so that the closer two-dimensional images are extracted as the first region.

The methods described above are just examples. Therefore, modifications can be made to the above-described methods. For instance, the control unit may change the method of extracting the first region based on conditions or situations where the three-dimensional image is captured (e.g., capturing site, capturing method, and capturing angle, among others). Additionally, the control unit may change the method of extracting the first region depending on the processing capability of the control unit of the medical image processing device.

The medical image processing method exemplified in this disclosure is executed in a medical image processing system that processes data of a three-dimensional image of a biological tissue. The medical image processing system includes a first image processing device and a second image processing device connected to each other via a network. The medical image processing method includes an image acquisition step, an extraction step, a transmission step, and a first structure detection step. In the image acquisition step, the first image processing device acquires a three-dimensional image of the tissue. In the extraction step, the first image processing device extracts a first region, which is a part of the three-dimensional image. In the transmission step, the first image processing device transmits the first region extracted at the extraction step to the second image processing device. In the first structure detection step, the second image processing device inputs the first region into a mathematical model and obtains detection results of a specific structure in the first region. This mathematical model is trained by a machine learning algorithm and is configured to output detection results of a specific structure in the tissue depicted in the input image.

In this case, even if the program to run the mathematical model trained by the machine learning algorithm is not embedded in the first image processing device, as long as the first and second image processing devices are connected via a network, the aforementioned processes can be executed appropriately.

The specific configurations of the first and second image processing devices may be chosen as appropriate. For instance, the first image processing device may be at least one of a PC, a mobile terminal, and a medical imaging device. The first image processing device may be placed in a facility that conducts diagnosis or examination of a subject. Additionally, the second image processing device may be a server (for example, a cloud server).

Furthermore, the second image processing device may execute an output step to output the detection results of the first structure detection step to the first image processing device. The first image processing device may execute a second structure detection step to detect a specific structure in the second region—a region that was not extracted as the first region in the extraction step—based on the detection results of the specific structure from the first region outputted by the mathematical model. In this scenario, both the first and second structure detection processes are properly executed within the medical image processing system.

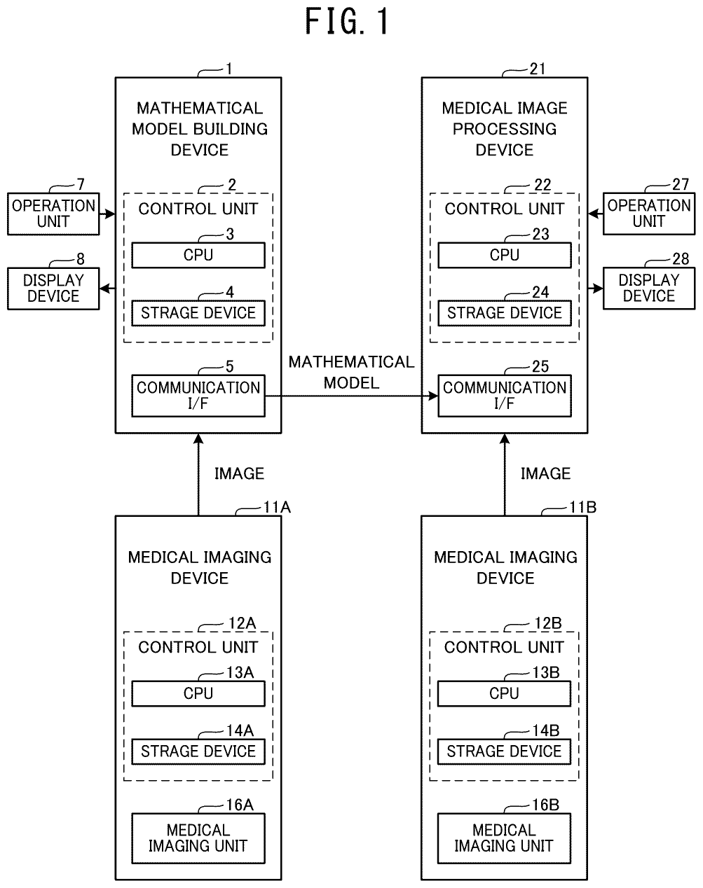

Hereinafter, a typical embodiment in this disclosure will be described with reference to the drawings. As shown in FIG. 1, in this embodiment, a mathematical model building device 1, a medical image processing device 21, and medical imaging devices 11A and 11B are used. The mathematical model building device 1 builds a mathematical model by training a model using a machine learning algorithm. The built mathematical model outputs a detection result of a specific structure (e.g., a layer, a boundary of layers, or the like) of a tissue in the input image. The medical image processing device 21 detects the specific structure of the tissue in the image using the mathematical model. The medical imaging devices 11A and 11B capture images of living tissue (in this embodiment, the retinal tissue of the subject eye).

As an example, in this embodiment, a personal computer (hereinafter referred to as a “PC”) is used for the mathematical model building device 1. Details will be described later, but the mathematical model building device 1 builds the mathematical model by training the model using images (hereinafter referred to as “input data”) obtained from the medical imaging device 11A and outputs data indicating the specific structure of the tissue in the input data. However, the device configured to serve as the mathematical model building device 1 is not necessarily limited to a PC. For example, the medical imaging device 11A may serve as the mathematical model building device 1. Additionally, controlling parts of multiple devices (for example, a CPU of the PC and a CPU13A of the medical imaging device 11A) may collaborate to produce the mathematical model.

In addition, a PC is used for the medical image processing device 21 in this embodiment. However, the device that is configured to serve as the medical image processing device 21 is not necessarily limited to a PC. For example, the medical imaging device 11B or a server may function as the medical image processing device 21. When the medical imaging device (in this embodiment, an OCT device) 11B serves as the medical image processing device 21 as well, the medical imaging device 11B can capture a three-dimensional image of the biological tissue and detect the specific structure in the tissue from the captured three-dimensional image. Furthermore, a mobile device such as a tablet device or smartphone may also function as the medical image processing device 21. Controlling parts of multiple devices (e.g., the CPU of the PC and the CPU13B of the medical imaging device 11B) can collaborate to carry out various processes.

Next, the mathematical model building device 1 will be described below. For example, the mathematical model building device 1 may be located in a facility of a manufacturer (a maker) or another entity that provides users with the medical image processing device 21 or medical image processing programs. The mathematical model building device 1 is equipped with a control unit 2 that carries out various control processes and a communication I/F 5. The control unit 2 includes a CPU3, which is configured to perform controlling, and a storage device 4, which is configured to store programs, data, and the like. The storage device 4 stores a mathematical model building program for executing a mathematical model building process, as will be described later. Moreover, the communication I/F 5 connects the mathematical model building device 1 to other devices (e.g., the medical imaging device 11A and the medical image processing device 21).

The mathematical model building device 1 is connected to an operation unit 7 and a display device 8. The operation unit 7 is operated by users to input various instructions into the mathematical model building device 1. As the operation unit 7, at least one of, for instance, a keyboard, mouse, touch panel, or the like may be used. Along with, or in place of, the operation unit 7, a microphone or similar device may also be used to input various instructions. The display device 8 shows various images. A variety type of devices capable of displaying images (e.g., monitors, displays, projectors, etc.) can be used as the display device 8. In this disclosure, the term “image” includes both static images and moving images (i.e., movies).

The mathematical model building device 1 acquires image data (hereinafter, simply referred to as an “image”) from the medical imaging device 11A. The mathematical model building device 1 obtains the image data from the medical imaging device 11A by means such as wired communication, wireless communication, or detachable storage media (for example, a USB memory).

Next, the medical image processing device 21 will be described below. The medical image processing device 21, for instance, is placed in a facility (e.g., a hospital or health checkup facility) that conducts diagnoses or examinations for subjects. The medical image processing device 21 is equipped with a control unit 22 that performs various control processes and a communication I/F 25. The control unit 22 includes a CPU23, which is configured to perform controlling, and a storage device 24, which is configured to store programs, data, and the like. Stored in the storage device 24 is a medical image processing program for executing medical image processing processes (first to fifth detection processes). The medical image processing program includes a program that implements the mathematical model built by the mathematical model building device 1. The communication I/F 25 connects the medical image processing device 21 to other devices (e.g., the medical imaging device 11B and the mathematical model building device 1).

The medical image processing device 21 is connected to an operation unit 27 and a display device 28. As the operation unit 27 and the display device 28, various devices can be used as with the operation unit 7 and the display device 8 for the mathematical model building deice 1.

The medical imaging device 11 (11A, 11B) is equipped with a control unit 12 (12A, 12B) that performs various control processes and a medical imaging unit 16 (16A, 16B). The control unit 12 consists of a controller (i.e., a CPU 13 (13A, 13B)) and a storage device 14 (14A, 14B) that is configured to store programs, data, and the like.

The medical imaging unit 16 is equipped with various components necessary for capturing images of biological tissues (in this embodiment, ophthalmic images of the subject eye). The medical imaging unit 16 in this embodiment includes an OCT light source, an optical element that divides emitted OCT light from the OCT light source into measurement light and reference light, a scanning unit to scan the measurement light, an optical system to emit the measurement light on the subject eye, and a photo-receiving element that receives composite light of the light reflected by the tissue and the reference light.

The medical imaging device 11 can capture two-dimensional tomographic images and three-dimensional tomographic images of a biological tissue (in this embodiment, the fundus of the subject eye). In detail, the CPU 13 captures a two-dimensional tomographic image of the cross-section intersecting the scan line by scanning the tissue with the OCT light (measurement light) along the scan line. The two-dimensional tomographic image may be an averaged image generated by performing an additive averaging process on multiple tomographic images on the same part of the tissue. Also, the CPU 13 captures a three-dimensional tomographic image of the tissue by scanning the tissue with the OCT light in two dimensions. For example, the CPU 13 captures multiple two-dimensional tomographic images by scanning the tissue with the measurement light along multiple scan lines at different positions within a two-dimensional area when the tissue is viewed from the front side thereof. Thereafter, the CPU 13 obtains a three-dimensional tomographic image by combining the captured multiple two-dimensional tomographic images, which will be described later more detail.

(Mathematical Model Building Process)

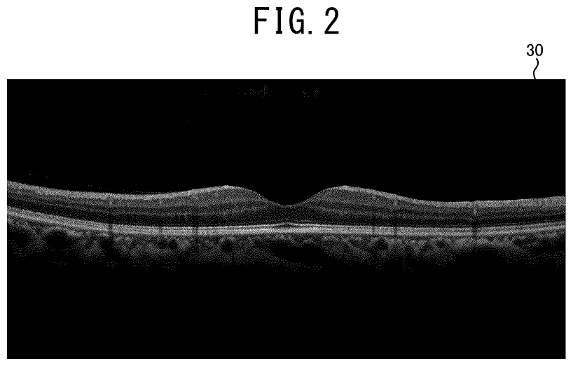

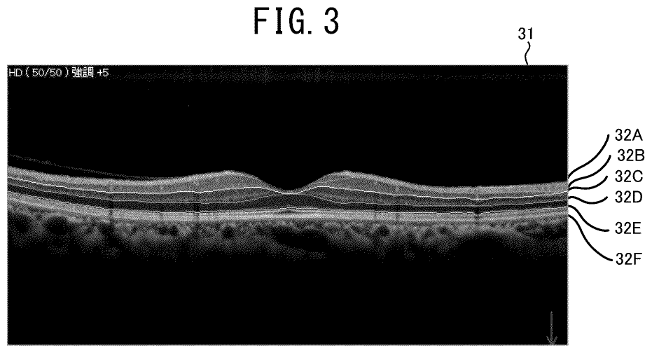

Referring to FIGS. 2 and 3, a mathematical model building process executed by the mathematical model building device 1 will be described. The mathematical model building process is executed by the CPU 3 according to the mathematical model building program stored in the storage device 4. In the mathematical model building process, a mathematical model is trained with multiple types of training data to build a model that is configured to output a detection result of a specific structure in a tissue captured in images. The training data includes input and output data.

First, the CPU 3 acquires, as input data, data of training images that are captured by the medical imaging device 11A. In this embodiment, the training image data is acquired by the mathematical model building device 1 after the medical imaging device 11A generated the training image data. However, the CPU 3 may obtain signals (e.g., OCT signals) that serve as the basis for generating training images from the medical imaging device 11A and generate the training images based on the obtained signals to acquire the training image data.

In this embodiment, the tissue structure as a detection target from images is a layer of the fundus tissue of the subject eye and/or a boundary of layers of the fundus tissue (hereinafter simply referred to as a “layer/boundary”). In this case, images of the fundus tissue of the subject eye are acquired as training images. Specifically, in the mathematical model building process, the type of the training images may be selected depending on the type of the images that will be input into the mathematical model to detect the structure from the images by the medical image processing device 21. For instance, if the image input into the mathematical model to detect the structure (the layer/boundary of the fundus) is a two-dimensional image (a two-dimensional tomographic image of the fundus), then in the mathematical model building process, a two-dimensional image (a two-dimensional tomographic image of the fundus) is used as a training image. FIG. 2 shows an example of a training image 30, which is a two-dimensional tomographic image of a fundus. The training image 30 illustrated in FIG. 2 shows multiple layers/boundaries in the fundus.

On the contrary, if the image input into the mathematical model to detect the structure (layers/boundaries of the fundus) is a one-dimensional image (for instance, an A-scan image that extends in one direction along the optical axis of the OCT measurement light), then in the mathematical model building process, a one-dimensional image (A-scan image) is used as a training image.

Next, the CPU 3 acquires the output data indicating a specific structure of the tissue captured in the training image. FIG. 3 shows an example of the output data 31 that indicates a specific boundary when a two-dimensional tomographic image of the fundus is used as the training image 30. The output data 31 illustrated in FIG. 3 contains data of labels 32A to 32F that indicate positions of six boundaries of the fundus tissue captured in the training image 30 (refer to FIG. 2). In this embodiment, the data of the labels 32A to 32F in the output data 31 is generated when an operator operates the operation unit 7 while looking at the boundaries in the training image 30. However, the method for generating the label data may also be changed. Note that if the training image is a one-dimensional image, the output data would be data that indicates the position of a specific structure in the one-dimensional image.

Next, the CPU 3 executes training of the mathematical model using the training data via a machine learning algorithm. As for the machine learning algorithm, examples such as neural networks, random forests, boosting, and support vector machines (SVM) are generally used.

Neural networks are methods where the behavior of biological neural networks is mimicked. Types of neural networks include, for instance, feedforward neural networks, RBF networks (Radial Basis Function), spiking neural networks, convolutional neural networks, recurrent neural networks (like RNNs, feedback neural networks, etc.), and probabilistic neural networks (like Boltzmann machines, Bayesian networks, etc.).

Random forests are methods that learn based on randomly sampled training data, and as a result, generate numerous decision trees. When using random forests, several pre-trained decision trees are navigated through their branches, and the average outcome (or majority vote) from each decision tree is taken.

Boosting is a method that generates a strong classifier by combining multiple weak classifiers. By sequentially training simple and weak classifiers, a strong classifier is produced.

SVM (Support Vector Machines) are a method that builds a two-class pattern recognizer using linear input elements. For instance, SVM learns the parameters of the linear input elements based on a criterion which seeks a hyperplane that maximizes the margin (distance) between it and each data point from the training data (known as the hyperplane separation theorem).

The mathematical model refers, for instance, to a data structure used to predict the relationship between input and output data. The mathematical model is built by being trained using training data. As previously mentioned, training data consists of pairs of input and output data. For example, through training, correlation data (like weights) between each input and output is updated.

In this embodiment, a multilayer neural network is used as the machine learning algorithm. The neural network includes an input layer for data input, an output layer for generating predicted data, and one or more hidden layers between the input and output layers. Each layer consists of multiple nodes (also referred to as units). Specifically, in this embodiment, a type of multilayer neural network called a Convolutional Neural Network (CNN) is used. However, other machine learning algorithms may also be used. For example, a Generative Adversarial Network (GAN), which uses two competing neural networks, may also be used as the machine learning algorithm. The program and data realizing the built mathematical model are integrated into the medical image processing device 21.

(Three-Dimensional Image)

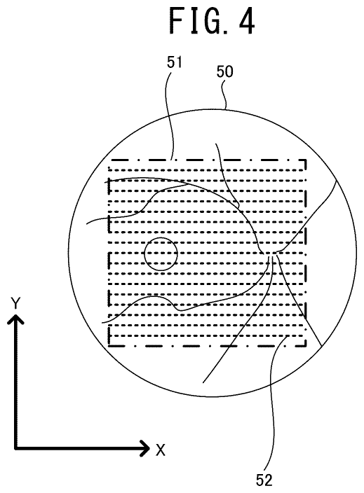

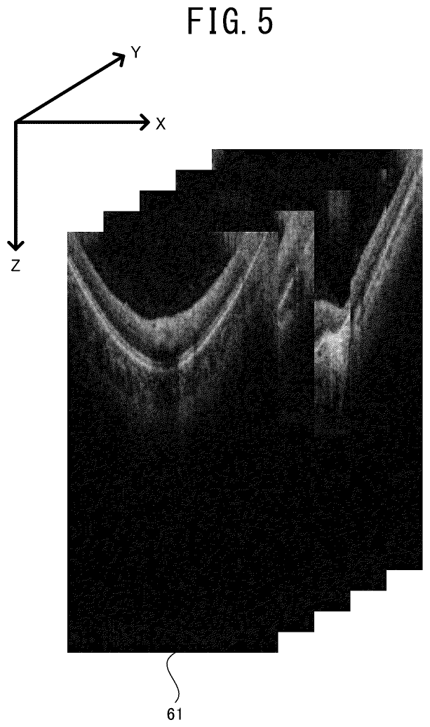

Referring to FIGS. 4 and 5, an example of a three-dimensional image, which is a target image from which a tissue structure is detected, is described. As shown in FIG. 4, the medical imaging device 11B of this embodiment scans the tissue with light (measurement light) within a two-dimensional region 51 of the biological tissue 50 (for example, the retinal tissue shown in FIG. 4). Specifically, the medical imaging device 11B of this embodiment captures a two-dimensional image 61 (see FIG. 5) that extends in Z-direction along the light axis and in X-direction perpendicular to Z-direction by scanning the tissue with light along the scan line 52 extending in a predetermined direction within the region 51. In the example shown in FIG. 4, Z-direction corresponds to the direction perpendicular to the two-dimensional region 51 (i.e., a depth direction), and X-direction corresponds to the direction in which the scan line 52 extends. Subsequently, the medical imaging device 11B changes the position of the scan line 52 in Y-direction within the region 51 and repeatedly captures the two-dimensional image 61. Y-direction is a direction that intersects both Z and X-directions (perpendicularly intersecting in this embodiment). As a result, multiple two-dimensional images 61 that pass through each of the multiple scan lines 52 and extend in the depth direction of the tissue are captured. Then, as shown in FIG. 5, by arranging the multiple two-dimensional images 61 in Y-direction (i.e., the direction intersecting each of the two-dimensional image areas), a three-dimensional image in the region 51 is generated.

The following describes the first to fifth detection processes performed by the medical image processing device 21 of this embodiment. In the first to fifth detection processes, a specific structure of the tissue appearing in the three-dimensional image is detected. In this embodiment, the medical image processing device 21, which is a PC, acquires a three-dimensional image from the medical imaging device 11B and detects the specific structure of the tissue in the acquired three-dimensional image. However, as previously mentioned, other devices may also function as the medical image processing device. For instance, the medical imaging device (in this embodiment, an OCT device) 11B itself can execute the first to fifth detection processes that will be described below. Also, multiple control units can collaboratively execute the first to fifth detection processes. In this embodiment, the CPU 23 of the medical image processing device 21 executes the first to fifth detection processes in accordance with the medical image processing program stored in the storage device 24.

(First Detection Process)

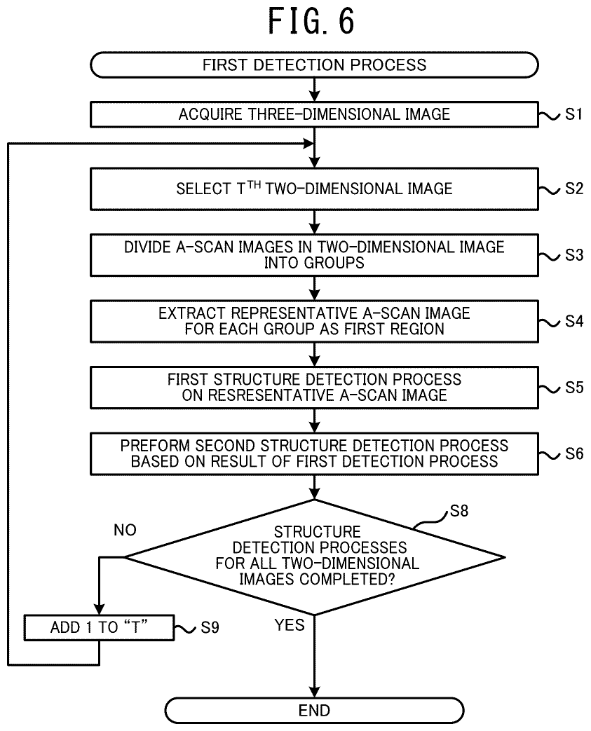

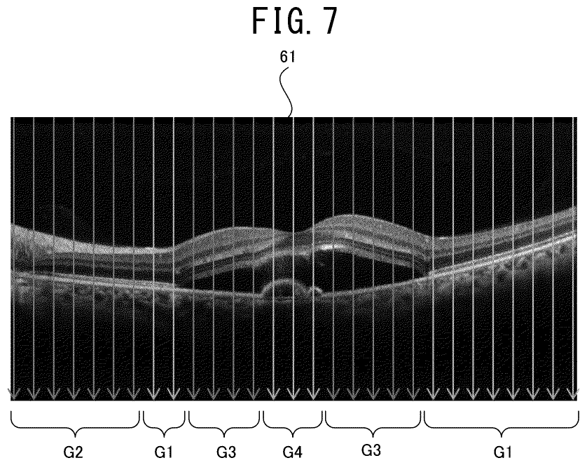

Referring to FIGS. 6 and 7, the first detection process is described. In the first detection process, the structure is detected from each two-dimensional image 61 based on a row of pixels that constitute the two-dimensional image 61 as a unit of processing.

As shown in FIG. 6, when the CPU 23 starts the first detection process, the CPU 23 acquires a three-dimensional image that is a target from which a specific structure is detected (S1). For example, a user operates the operation unit 27 (refer to FIG. 1) to select a three-dimensional image from multiple three-dimensional images as a detection target for the specific structure. The CPU 23 then acquires data of the three-dimensional image selected by the user.

The CPU 23 selects the T^{th} (T is a natural number, initially set as 1) two-dimensional image 61 among the multiple two-dimensional images 61 that constitute the three-dimensional image (S2). In this embodiment, each of the multiple two-dimensional images 61 that constitute the three-dimensional image is numbered in an order in which the images 61 are arranged in Y-direction. During the process of S2, the multiple two-dimensional images 61 are selected in the order from the one located on the outermost side of the two-dimensional images 61 in Y-direction.

The CPU 23 classifies multiple A-scan images in the two-dimensional image 61 selected at S2 into multiple groups (S3). As shown in FIG. 7, the two-dimensional image 61 captured by the OCT device is formed of multiple A-scan images indicated by arrows in FIG. 7. Each A-scan image consists of a row of pixels that extends in the direction along the optical axis of the OCT measurement light. At S3, the CPU 23 classifies the multiple A-scan images with high similarity to each other into the same group, regardless of their positions. In the example shown in FIG. 7, the group G1 includes the A-scan images from areas where no layer separation exists and the retinal nerve fiber layer is thin. The group G2 includes the A-scan images from an area without layer separation and with a thick retinal nerve fiber layer. The group G3 includes the A-scan images from areas where the IS/OS line is separated. The group G4 includes the A-scan images from an area where both the IS/OS line and the retinal pigment epithelium layer are separated.

Next, the CPU 23 extracts a representative A-scan image, which represents a row of pixels for each of the multiple groups, as a first region (S4). The first region refers to an area within the three-dimensional image where the specific structure is detected using the mathematical model trained by the machine learning algorithm. The method to extract the representative A-scan image from the multiple A-scan images in a group may be chosen appropriately. In this embodiment, the CPU 23 extracts, as the representative A-scan image, a row of pixels that is obtained by performing an additive average processing on the multiple A-scan images classified into each of the groups. As a result, the representative A-scan image that accurately represents the corresponding group is properly extracted.

The CPU 23 executes a first structure detection process on the representative A-scan image (the first region) extracted from each group (S5). The first structure detection process is a process to detect a specific structure using a mathematical model. In other words, when executing the first structure detection process, the CPU 23 inputs the first region extracted from the three-dimensional image (the representative A-scan image in the example shown in FIG. 6) into a mathematical model trained by a machine learning algorithm. The mathematical model outputs a detection result of the specific structure (in this embodiment, layers or boundaries in the fundus) within the first region. The CPU 23 retrieves the detection result outputted by the mathematical model. While the computational load for the first structure detection process is high as compared to a traditional image processing method, the specific structure can be detected within the image with high accuracy.

The CPU 23 selects the A-scan images from each group that were not extracted as the first region (in this embodiment, the representative A-scan image) as a second region and executes a second structure detection process for each of the groups (S6). The second structure detection process is a process to detect, based on the detection result of the first structure detection process, a specific structure within the second region that was not selected as the first region out of the entire area of the three-dimensional image. The computational load for the second structure detection process is lower than that of the first structure detection process. Furthermore, the second structure detection process is executed based on the result of the first structure detection process with high accuracy. Therefore, the structure of the second region is accurately detected as well.

In more detail, at the step of S6 in this embodiment, the CPU 23 provides the detection result of the structure in the second region by comparing the detection result and pixel information of each of the pixels constituting the first region (i.e., the representative A-scan image) with pixel information of each of pixels constituting the second region. Here, the CPU 23 may consider the positional relationship (for instance, proximity in Z-direction) between each of the pixels constituting the second region and each of the pixels constituting the first region (the representative A-scan belonging to the same group). Alternatively, the CPU 23 may also perform the second structure detection process for the second region by interpolation processing using the result of the first structure detection process.

As described at the step of S3, the degree of similarity between the multiple A-scan images classified into the same group is high. Therefore, at S5 and S6 of this embodiment, by executing the first and second structure detection processes for each group, the accuracy of the second structure detection process is further improved.

The CPU 23 determines whether the structure detection processes for all the two-dimensional images have been completed (S8). If not (S8: NO), the counter T, which indicates the order assigned to the two-dimensional image, is incremented by “1” (S9), and the process returns to S2. When the structure detection processes for all the two-dimensional images are completed (S8: YES), the first detection process ends.

In the first detection process of this embodiment, multiple rows of the pixels (i.e., A-scan images) that constitute a two-dimensional image are classified into multiple groups, and the first region is extracted from each of the groups. However, the method of extracting the first region may be changed. For example, the CPU 23 may classify a small region (patch) formed of multiple rows of pixels (for example, the A-scan images) into multiple groups, and extract the first region for each of the groups. Alternatively, the CPU 23 may extract the first regions from multiple rows of pixels constituting a two-dimensional image at regular intervals.

(Second Detection Process)

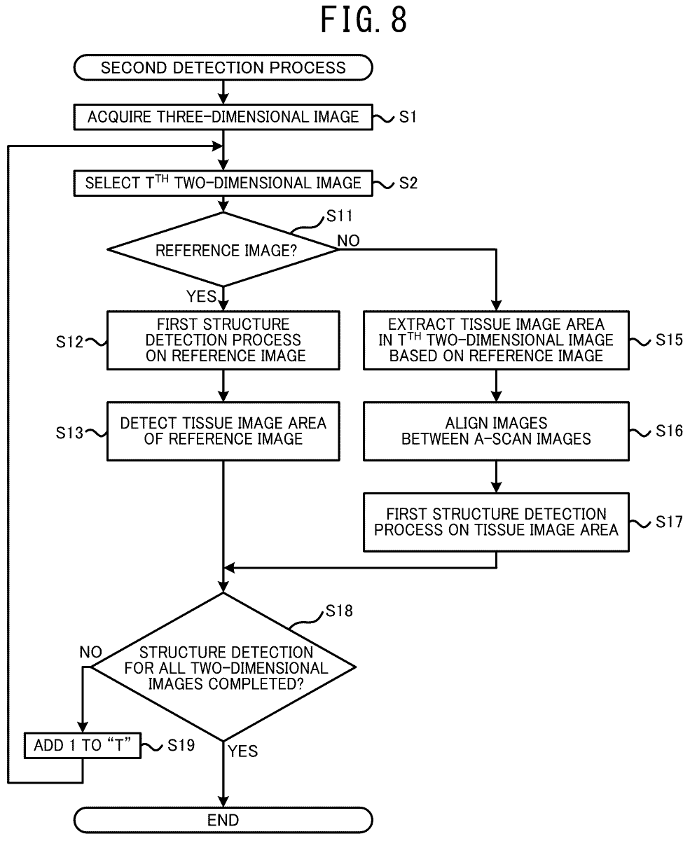

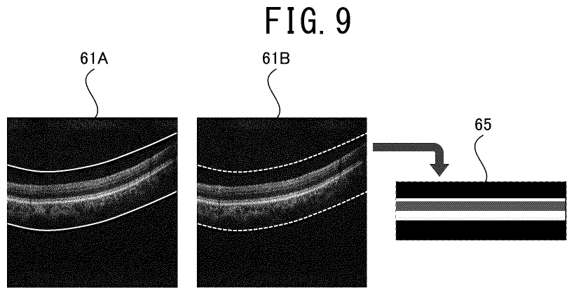

Referring to FIGS. 8 and 9, the second detection process will be described. In the second detection process, an image area where the tissue structure is captured is extracted from each two-dimensional image. The first structure detection process using a mathematical model is performed on the extracted image area. Therefore, areas where no tissue image is captured are excluded from a target area from which the specific tissue is detected by the mathematical model. Note that among the second to fifth detection processes, steps similar to those described in the previously mentioned first detection process are simply described in the explanation.

As shown in FIG. 8, when the CPU 23 starts the second detection process, the CPU 23 acquires a three-dimensional image which is a detection target for the specific structure (S1). The CPU 23 selects the T^{th} two-dimensional image 61 from the multiple two-dimensional images 61 that constitute the acquired three-dimensional image (S2).

Next, the CPU 23 determines whether to use the T^{th} two-dimensional image 61 as a reference image 61A (refer to FIG. 9) (S11). The reference image 61A is an image that serves as a basis for extracting image areas from other two-dimensional images 61B. The method to select the reference image 61A among from the multiple two-dimensional images 61 may be appropriately chosen. As an example, in this embodiment, the CPU 23 selects the reference images 61A from the multiple two-dimensional images 61 at regular intervals. Note that in the first time at S11, the 1^{st} (i.e., T=1) two-dimensional image 61 is always selected as the reference image 61A.

When the T^{th} two-dimensional image 61 is set as the reference image 61A (S11: YES), the CPU 23 performs the first structure detection process on the reference image 61A (i.e., the T^{th} two-dimensional image 61) (S12). In other words, the CPU 23 inputs the reference image 61A into a mathematical model and obtains a detection result of the specific structure in the tissue shown in the reference image 61A.

Next, based on the structure detection result obtained at S12, the CPU 23 identify an image area in the reference image 61A where the tissue image is captured (S13). As previously mentioned, at the first structure detection process using the mathematical model, the specific structure is likely to be detected with high accuracy. Therefore, the image area detected based on the result obtained at S12 can be also identified with high accuracy. In the reference image 61A shown in FIG. 9, the area enclosed by two solid lines is detected as the image area based on the result of the structure detection process (in this embodiment, the detection result of the layers and boundaries of the retina).

On the other hand, if the selected T^{th} two-dimensional image 61 is not set as the reference image 61A (S11: NO), the CPU 23 extracts the image area of the T^{th} two-dimensional image 61B (refer to FIG. 9) as the first region based on the already detected image area of the reference image 61A (S15). As a result, the image area of the T^{th} two-dimensional image 61B is detected with lower amount of computational work as compared with using the mathematical model. In the example shown in FIG. 9, the area enclosed by two broken lines of the two-dimensional image 61B, which is located near the reference image 61A, is detected as the image area.

It should be noted that at S15 of this embodiment, the CPU 23 detects the image area of the two-dimensional image 61B by comparing the detection result of the image area and pixel information for each of pixels constituting the reference image 61A with pixel information of each of pixels constituting the two-dimensional image 61B. Additionally, the CPU 23 also considers the positional relationship (in this embodiment, X-Z coordinates relationship) between each of the pixels constituting the reference image 61A and each of the pixels constituting the two-dimensional image 61B when detecting the image area of the two-dimensional image 61B.

Next, the CPU 23 aligns the position of the tissue images (in this embodiment, aligns the positions in Z-direction) between multiple rows of pixels (in this embodiment, the previously mentioned multiple A-scan images) constituting the T^{th} two-dimensional image 61B (S16). For instance, by aligning the positions of the tissue image area of the two-dimensional image 61B with respect to the reference image 61, the CPU 23 makes the shape (curved shape) of the tissue image area of the two-dimensional image 61B and the reference image similar to each other. With this state, by cutting out the curved shape to be flat or by shifting the A-scan image in Z-direction, the CPU 23 makes the tissue image area 65 extracted from the two-dimensional image 61B rectangular (or substantially rectangular). In other words, through the second detection process, the tissue image fits appropriately within a rectangular image area 65 (the first region), and the size of the rectangular image area 65 is likely to be reduced. The CPU 23 then performs the first structure detection process on the rectangular image area 65 (S17). In other words, the CPU 23 obtains the detection result of the specific structure in the image area 65 by inputting the rectangular image area 65 into the mathematical model.

The CPU 23 determines whether the structure detection processes for all the two-dimensional images 61 have been completed (S18). If not (S18: NO), the counter T indicating the order assigned to the two-dimensional images 61 is incremented by “1” (S19), and the process returns to S2. When the structure detection processes for all the two-dimensional images 61 are complete (S18: YES), the second detection process ends. In the second detection process, the final detection result is obtained by adding the inverse of the amount of movement in the alignment that was executed for each A-scan image at S16 to the structure detection result obtained at S17.

During the second detection process of this embodiment, the tissue image area of the two-dimensional image 61B is extracted based on the tissue image area of the reference image 61A. However, the method of extracting the tissue image area may be changed. For instance, the CPU 23 may identify the tissue image area by performing a known-image processing on the two-dimensional image 61.

(Third Detection Process)

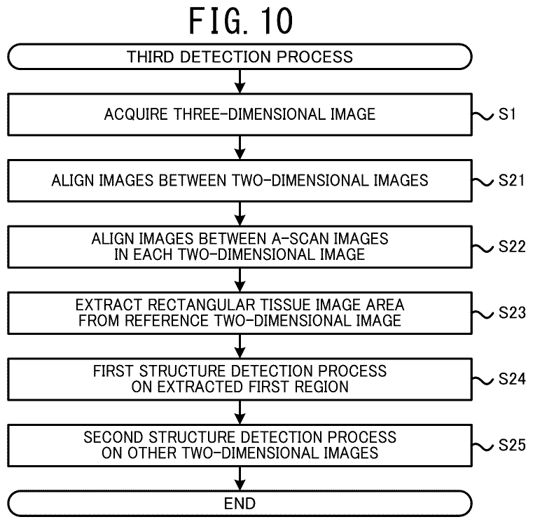

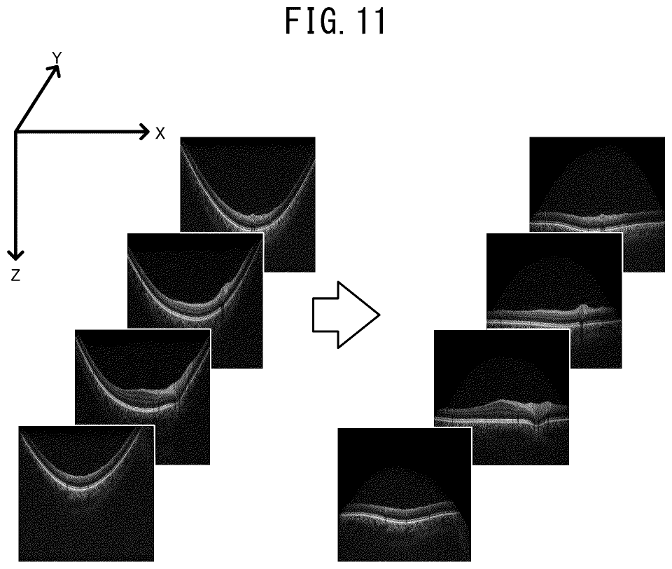

Referring to FIGS. 10 and 11, the third detection process will be described. In the third detection process, for the multiple two-dimensional images 61 that constitute the three-dimensional image, aligning images within each two-dimensional image 61 and aligning tissue images between the two-dimensional images 61 are executed. Thereafter, the tissue image areas are extracted, and a specific structure in the extracted image areas is detected.

As shown in FIG. 10, when the CPU 23 starts the third detection process, the CPU 23 acquires the three-dimensional image which is a detection target for the specific structure (S1). The CPU 23 executes the alignment of tissue images (in this embodiment, alignment in Z-direction) between the multiple two-dimensional images 61 that constitute the three-dimensional image (S21). Further, for each of the two-dimensional images 61 that constitute the three-dimensional image, the CPU 23 executes the alignment of the tissue images (in this embodiment, alignment in Z-direction) between multiple rows of pixels (in this embodiment, the above-described multiple A-scan images) that constitute the two-dimensional image 61 (S22).

At S22 of this embodiment, the CPU 23 creates multiple two-dimensional images each of which spreads in Y-Z direction. Through the alignment of the tissue images between the created two-dimensional images, the CPU 23 executes the alignment of adjacent pixels in the two-dimensional images 61 that spread in X-Z direction. As a result, negative effects by noise, etc., can be reduced as compared to performing the alignment between multiple A-scan images. Note that the order of steps of S21 and S22 can be reversed.

FIG. 11 a comparison between two-dimensional images before conducting the alignment of the tissue images and two-dimensional images after conducting the alignment of the tissue images (the alignment includes both the alignment between the two-dimensional images 61 and the alignment within each two-dimensional image 61). The left side of FIG. 11 shows the two-dimensional images before conducting the alignment, while the right side shows the two-dimensional images after conducting the alignment. As shown in FIG. 11, due to the alignment within each image and between the multiple images, the position of the tissue image in each two-dimensional image is similar to each other.

Next, the CPU 23 selects at least one of the multiple two-dimensional images 61 that constitute the three-dimensional image as a reference image. From the two-dimensional image 61 selected as the reference image, the CPU 23 extracts a rectangular image area as the first region (S23). The method of selecting the reference image from among the multiple two-dimensional images 61 can be chosen as described at S11. In this embodiment, the CPU 23 selects the reference images at regular intervals from the multiple two-dimensional images 61. The multiple two-dimensional images 61 not selected as the reference image serve as the second region on which the structure detection process using the mathematical model is not executed.

The CPU 23 executes the first structure detection process on the first region extracted at S23 (S24). That is, by inputting the first region extracted at S23 into the mathematical model, the CPU 23 obtains a detection result of the specific structure in the first region.

Furthermore, the CPU 23 performs the second structure detection process on the two-dimensional images 61 (i.e., the second region) that were not selected as the reference image (S25). That is, the CPU 23 detects the specific structure in the second region based on the result of the first structure detection process on the first region that is the reference image. Here, in the third detection process, the image alignment between the multiple two-dimensional images 61 was performed at S21. Therefore, at S25, by performing comparison between pixels having close coordinates (in this embodiment, X-Z coordinates) between the first and second regions, the structure in the second region can be appropriately detected.

Note that in the third detection process, the signs (plus, minus) of the movement amounts of the alignments executed for each of the A-scan images at S21 and S22 are inverted, and this inversion is added to the detection results obtained at S24 and S25 to acquire the final structure detection result.

Modifications to the steps S23 to S25 in the third detection process will be described. For instance, after performing the image alignment for the entire three-dimensional image at S21 and S22, the CPU 23 may extract a rectangular (or substantially rectangular) image area from the three-dimensional image and execute the first structure detection process on this extracted image area. In this case, the CPU 23 may calculate the average of all the A-scan images from the three-dimensional image that ware aligned at S21 and S22 and identify the range of the image from the averaged A-scan image. Then, based on the identified image range, the CPU 23 may extract the rectangular image area from each two-dimensional image 61, and by inputting this extracted image area into the mathematical model, the CPU 23 may perform the first structure detection process. In this case, the first structure detection process may be omitted. In this modified example, since the first structure detection process is only executed for the area where an image is likely to exist, computation amount during the processing can be reduced.

(Fourth Detection Process)

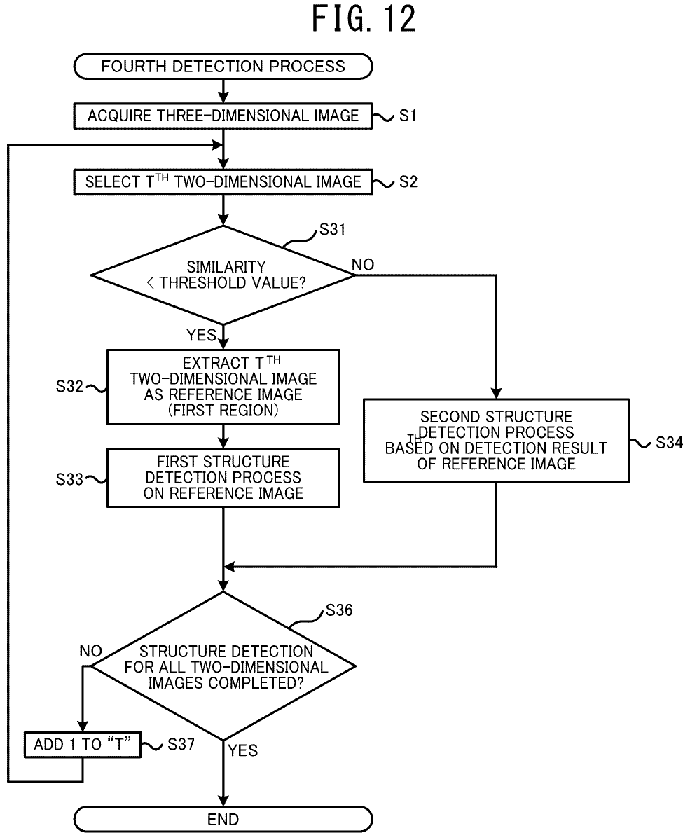

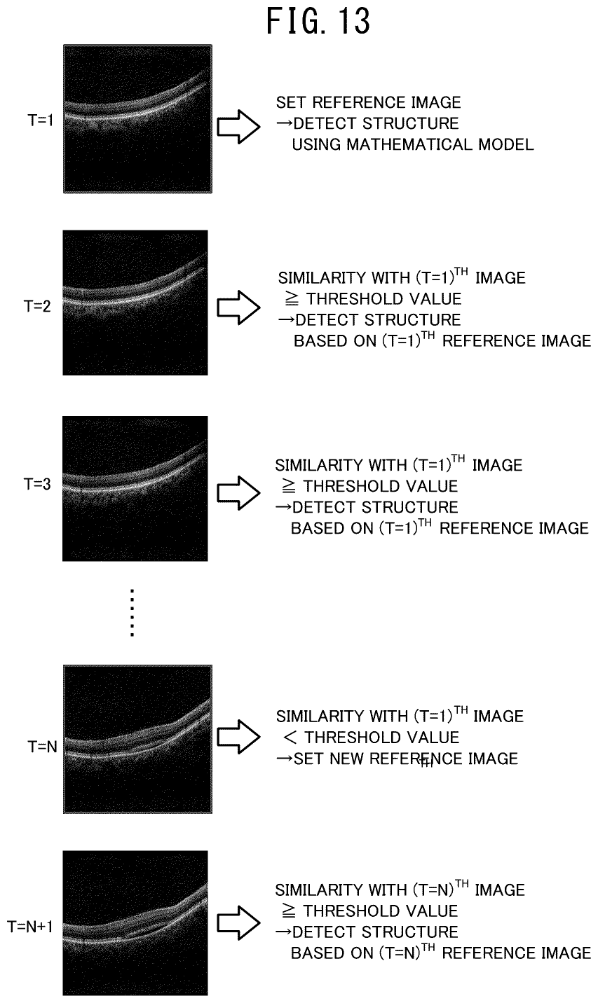

Referring to FIGS. 12 and 13, the fourth detection process will be described. In the fourth detection process, some of the multiple two-dimensional images 61 that constitute the three-dimensional image are extracted as the first region that is a target for the first structure detection process. Specifically, based on the degree of similarity between the two-dimensional images 61, some of the two-dimensional images 61 are extracted as the first region.

As shown in FIG. 12, when the CPU 23 starts the fourth detection process, the CPU 23 acquires a three-dimensional image from which a specific structure is detected (S1). The CPU 23 then selects the T^{th} two-dimensional image 61 from the multiple two-dimensional images 61 that constitute the three-dimensional image (S2).

The CPU 23 determines whether the degree of similarity between a reference image at this timing and the T^{th} two-dimensional image 61 falls below a threshold value (S31). In the fourth detection process, the reference image serves as a criteria to determine whether other two-dimensional images 61 should be selected as either the first region or the second region. At the first time of S31, the reference image is not yet set. Thus, the process proceeds to S32 where the CPU 23 sets the (T=1)^{th} two-dimensional image 61 as the reference image and extracts the (T=1)th two-dimensional image 61 as the first region (S32). The CPU 23 then performs the first structure detection process on the (T=1)^{th} image, which is the reference image (S33).

On the other hand, if the degree of similarity between the reference image at this timing and the T^{th} two-dimensional image 61 is equal to or greater than the threshold value (S31: NO), the CPU 23 selects the T^{th} two-dimensional image 61 as the second region and performs the second structure detection process on the second region (S34). That is, the CPU 23 detects a specific structure in the T^{th} two-dimensional image 61 based on the result of the first structure detection process on the reference image, which has high similarity to the T^{th} two-dimensional image 61.

In general, the greater the distance (in this embodiment, the distance in Y-direction) between the T^{th} two-dimensional image 61 and the reference image, the more likely the degree of similarity between the two images decreases. Also, even if the distance between the T^{th} two-dimensional image 61 and the reference image is small, if the region has structural changes, the degree of similarity between the two images tends to decrease.

If the degree of similarity between the reference image this time and the T^{th} two-dimensional image 61 falls below the threshold value (S31: YES), the CPU 23 sets the T^{th} two-dimensional image 61 as a new reference image and extracts the T^{th} two-dimensional image 61 as the first region (S32). The CPU 23 then performs the first structure detection process on the T^{th} image that is selected as the new reference image (S33).

The CPU 23 determines whether the structure detection processes for all the two-dimensional images 61 have been completed (S36). If not (S36: NO), “1” is added to the counter T, which indicates the order assigned to each of the two-dimensional images (S37), and the process returns to S2. Once the structure detection process for all two-dimensional images is completed (S36: YES), the fourth detection process ends.

Referring to FIG. 13, the flow and advantageous effects of the fourth detection process will be described. First, the (T=1)^{th} two-dimensional image is set as the reference image. The (T=1)^{th} two-dimensional image is extracted as the first region and is the target image on which the first structure detection process using a mathematical model is performed.

In the example of FIG. 13, the (T=2)^{th} two-dimensional image is adjacent to the reference image (i.e., the (T=1)^{th} two-dimensional image). Also, the structural changes between the (T=1)^{th} and (T=2)^{th} two-dimensional images are small. As a result, the degree of similarity between the (T=2)^{th} two-dimensional image and the (T=1)^{th} two-dimensional image (i.e., the reference image) exceeds the threshold value. Thus, the specific structure in the (T=2)^{th} two-dimensional image 61 is detected based on the result of the first structure detection process on the (T=1)^{th} two-dimensional image. Note that in the example of FIG. 13, the degree of similarity between the (T=3)^{th} two-dimensional image and the (T=1)^{th} two-dimensional image (i.e., the reference image) also exceeds the threshold.

It is assumed that the degree of similarity between the (T=N)^{th} two-dimensional image and the (T=1)^{th} two-dimensional image (i.e., the reference image) falls below the threshold value. In this case, the (T=N)^{th} two-dimensional image is set as a new reference image and selected as a target image on which the first structure detection process using a mathematical model is performed. The process on the (T=N+1)^{th} two-dimensional image is executed using the (T=N)^{th} two-dimensional image as the reference image.

(Fifth Detection Process)

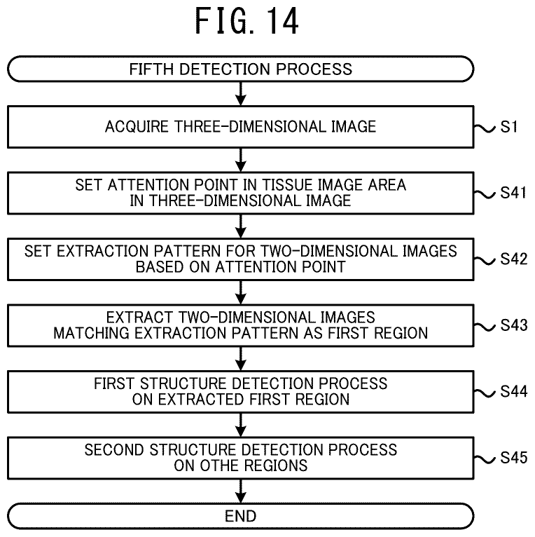

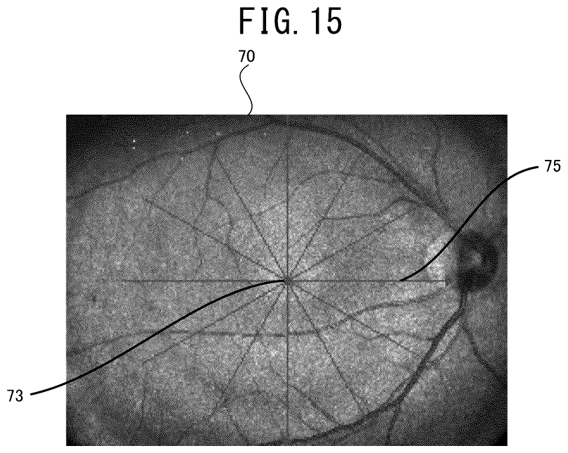

Referring to FIGS. 14 and 15, the fifth detection process will be described. In the fifth detection process, an attention point is set within the three-dimensional image area, and based on the set attention point, the first region is extracted.

As shown in FIG. 14, when the CPU 23 starts the fifth detection process, the CPU 23 obtains the three-dimensional image that is a target image from which a specific structure is detected (S1). The CPU 23 sets the attention point within the image area of the three-dimensional image (S41). For example, in this embodiment, the CPU 23 sets the attention point in accordance with instructions input by a user through the operation unit 27 (i.e., a position indicated by the user). Alternatively, the CPU 23 may detect a specific part in the three-dimensional image and set the detected specific part as the attention point. FIG. 15 shows a two-dimensional front image 70 viewed in a direction along the optical axis of the OCT measurement light in the imaging area of the three-dimensional image. In the example shown in FIG. 15, the macula is detected as a specific part of the examinee's retina, and the attention point 73 is set at the detected macula.

Next, the CPU23 sets an extraction pattern for multiple two-dimensional images based on the attention point (S42). The CPU23 extracts the two-dimensional images that match the set extraction pattern as the first region that is a detection target for the specific structure using a mathematical model (S43). The two-dimensional image extraction pattern set at S42 does not necessarily match each of the two-dimensional images 61 captured by the medical imaging device 11B, and may be set arbitrarily. For instance, in the example shown in FIG. when the three-dimensional image is viewed in a direction along the optical axis of the OCT measurement light, the extraction pattern 75 is set so that lines crossing the extracted two-dimensional image radially spread from the attention point 73. As a result, multiple two-dimensional images centered on the attention point 73 are extracted as the first region.

The CPU23 executes the first structure detection process on the first region extracted at S43 (S44). Also, for the second region of the three-dimensional image, which is a region other than the first region, the CPU23 executes the second structure detection process (S45). Since the first structure detection process and the second structure detection process are the same processes as described before, detailed explanations will be omitted.

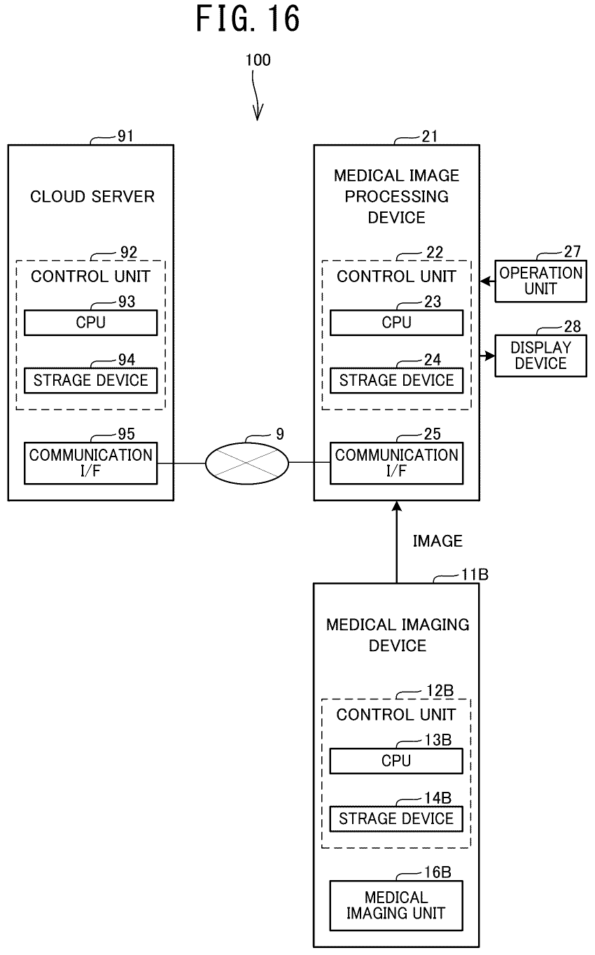

The technology disclosed in the above embodiment is just one example. Therefore, it is also possible to modify the technology exemplified in the above embodiment. Referring to FIG. 16, an explanation will be given regarding the system configuration of a medical image processing system 100, which is a modified example to the above-described embodiment. Note that for parts of the medical image processing system 100 that are similar to those described in the above embodiment (for instance, the medical image processing device 21 and the medical imaging device 11B, etc.), the same reference numerals as in the above embodiment are used, and their descriptions are omitted or simplified.

The medical image processing system 100 shown in FIG. 16 includes a medical image processing device 21 and a cloud server 91. The medical image processing device 21 processes data of a three-dimensional image taken by the medical imaging device 11B. Specifically, in the example shown in FIG. 16, the medical image processing device 21 serves as a first image processing device that executes processes (methods) other than the aforementioned first structure detection process (S5 in FIG. 6, S17 in FIG. 8, S24 in FIG. 10, S33 in FIG. 12, S44 in FIG. 14). However, a device different from the medical image processing device 21 (for example, the medical imaging device 11B, etc.) may also serve as the first image processing device.

The cloud server 91 is equipped with a control unit 92 and a communication I/F (interface) 95. The control unit 92 comprises a CPU 93, which acts as a controller, and a storage device 94 configured to store programs, data, and the like. The programs stored in the storage device 94 realize the aforementioned mathematical model. The communication I/F 95 connects the cloud server 91 and the medical image processing device 21 via a network (for example, the Internet) 9. In the example shown in FIG. 16, the cloud server 91 functions as a second image processing device that executes the aforementioned first structure detection process (S5 in FIG. 6, S17 in FIG. 8, S24 in FIG. 10, S33 in FIG. 12, S44 in FIG. 14).

The medical image processing device (the first image processing device) 21 executes a transmission step to transmit the first region extracted at S4 in FIG. 6, S15 in FIG. 8, S23 in FIG. 10, S32 in FIG. 12, and S43 in FIG. 14 to the cloud server 91. The cloud server 91 carries out the aforementioned first structure detection process. Additionally, the cloud server 91 executes an output step to output the results detected by the first structure detection process to the medical image processing device 21. As a result, even if the programs to run the mathematical model are not embedded in the medical image processing device 21, the various aforementioned processes are executed appropriately.

Furthermore, it is also possible to execute only a part of the processes exemplified in the above-described embodiment. For example, in the third detection process shown in FIG. 10, the first structure detection process (S24) for the first region and the second structure detection process (S25) for the other second region are executed. However, at S23, image areas may be detected from all the two-dimensional images 71 that constitute the three-dimensional image. In this case, the second structure detection process (S25) can be omitted.

Also, it is possible to combine and execute multiple processes exemplified in the first to fifth detection processes. For example, in the second detection process shown in FIG. 8, the second structure detection process for the area other than the image area has been omitted. However, it is also possible to execute the second structure detection process within the second detection process.

The process of acquiring a three-dimensional image at S1 in FIG. 6, FIG. 8, FIG. 10, FIG. 12, and FIG. 14 is an example of an “image acquisition step”. The process of extracting the first region at S4 in FIG. 6, S15 in FIG. 8, S23 in FIG. 10, S32 in FIG. 12, and S43 in FIG. 14 is an example of an “extraction step”. The first structure detection process shown in S5 in FIG. 6, S17 in FIG. 8, S24 in FIG. 10, S33 in FIG. 12, and S44 in FIG. 14 is an example of a “first structure detection step”. The second structure detection process shown in S45 in FIG. 6, S25 in FIG. 10, S34 in FIG. 12, and S45 in FIG. 14 is an example of a “second structure detection step”. The process of aligning the image within the two-dimensional image at S16 in FIGS. 8 and S21 in FIG. 10 is an example of a “two-dimensional image internal alignment step”. The process of aligning positions between multiple two-dimensional images at S22 in FIG. 10 is an example of a “multiple two-dimensional images alignment step”.

1. A medical image processing device that is configured to process data of a three-dimensional image of a biological tissue, the medical image processing device comprising
a controller configured to:
acquire, as an image acquisition step, a three-dimensional image of a tissue;
extract, as an extraction step, a first region from the acquired three-dimensional image, the first region being a part of the three-dimensional image; and
acquire, as a first structure detection step, a detection result of a specific structure of the tissue in the extracted first region by inputting the first region into a mathematical model that is trained by a machine learning algorithm to output a detection result of a specific structure that is shown in an image input into the mathematical model.

2. The medical image processing device according to claim 1, wherein
a second region is a region of the entire three-dimensional image that was not extracted as the first region at the extraction step, and
the controller is further configured to detect, as a second structure detection step, the specific structure in the second region based on the detection result of the specific structure in the first region that was output by the mathematical model.

3. The medical image processing device according to claim 1, wherein
in the extraction step, the controller is further configured to extract the first region from each of a plurality of two-dimensional images that constitute the three-dimensional image.

4. The medical image processing device according to claim 3, wherein
the controller is further configured to:
in the extraction step, divide a plurality of rows of pixels that constitute the two-dimensional image into a plurality of groups based on degree of similarity between the plurality of rows of pixels and extract, as the first region, a representative row of pixels representing each of the plurality of groups; and
in the first structure detection step, input the extracted representative row of pixels into the mathematical model.

5. The medical image processing device according to claim 3, wherein
the three-dimensional image is formed by arranging the plurality of two-dimensional images in a direction, and
in the extraction step, the controller is further configured to extract, as the first region, a tissue image area in which the tissue is shown from each of the plurality of two-dimensional images.

6. The medical image processing device according to claim 5, wherein
a reference image is defined as at least one of the plurality of two-dimensional images,
in the extraction step, the controller is further configured to:
detect a tissue image area in the reference image by inputting the reference image into the mathematical model; and
extract, as the first region, a tissue image area in at least another one of the plurality of two-dimensional images that is other than the reference image based on a detection result of the tissue image area in the reference image.

7. The medical image processing device according to claim 5, wherein
the controller is further configured to:
align, as a two-dimensional image internal alignment step, tissue images between a plurality of rows of pixels that constitute each of the plurality of two-dimensional images; and
in the first structure detection step, input, into the mathematical model, the first region having a rectangular shape that is subject to the two-dimensional image internal alignment step and the extraction step.

8. The medical image processing device according to claim 5, wherein
the controller is further configured to align, as a multiple two-dimensional images alignment step, tissue images between the plurality of two-dimensional images.

9. The medical image processing device according to claim 1, wherein
in the extraction step, the controller is further configured to extract, as the first region, one or some of the plurality of two-dimensional images included in the three-dimensional image.

10. The medical image processing device according to claim 9, wherein
the controller is further configured to:
perform the extraction step and the first structure detection step by setting, as the first region, a reference image that is one or some of the plurality of two-dimensional images included in the three-dimensional image; and thereafter
perform the extraction step and the first structure detection step by setting, as the first region, one or some of the plurality of two-dimensional images having degree of similarly with the reference image that is less than a threshold value.

11. The medical image processing device according to claim 9, wherein
the controller is further configured to, in the extraction step:
set an attention point in a tissue image area in the three-dimensional image;
set an extraction pattern for the plurality of two-dimensional images based on the set attention point; and
extract, as the first region, some of the plurality of two-dimensional images that match the set extraction pattern.

12. A non-transitory, computer readable, storage medium storing a medical image processing program for a medical image processing device configured to process data of a three-dimensional image of a biological tissue, the medical image processing program, when executed by a controller of the medical image processing device, causing the controller to perform:
acquiring, as an image acquisition step, a three-dimensional image of a tissue;
extracting, as an extraction step, a first region from the acquired three-dimensional image, the first region being a part of the three-dimensional image; and
acquiring, as a first structure detection step, a detection result of a specific structure of the tissue in the extracted first region by inputting the first region into a mathematical model that is trained by a machine learning algorithm to output a detection result of a specific structure that is shown in an image input into the mathematical model.

13. The storage medium according to claim 12, wherein
in the extraction step, the program further causes the controller to extract the first region from each of a plurality of two-dimensional images that constitute the three-dimensional image.

14. The storage medium according to claim 13, wherein
the three-dimensional image is formed by arranging the plurality of two-dimensional images in a direction, and
in the extraction step, the program further causes the controller to extract, as the first region, a tissue image area in which the tissue is shown from each of the plurality of two-dimensional images.

15. The storage medium according to claim 14, wherein
the program further causes the controller to align, as a multiple two-dimensional images alignment step, tissue images between the plurality of two-dimensional images.

16. A medical image processing method implemented by a medical image processing device configured to process data of a three-dimensional image of a biological tissue, the method comprising:
acquiring, as an image acquisition step, a three-dimensional image of a tissue;
extracting, as an extraction step, a first region from the acquired three-dimensional image, the first region being a part of the three-dimensional image; and
acquiring, as a first structure detection step, a detection result of a specific structure of the tissue in the extracted first region by inputting the first region into a mathematical model that is trained by a machine learning algorithm to output a detection result of a specific structure that is shown in an image input into the mathematical model.

17. The method according to claim 16, wherein
in the extraction step, the method further comprises extracting the first region from each of a plurality of two-dimensional images that constitute the three-dimensional image.

18. The method according to claim 17, wherein
the three-dimensional image is formed by arranging the plurality of two-dimensional images in a direction, and
in the extraction step, the method further comprises extracting, as the first region, a tissue image area in which the tissue is shown from each of the plurality of two-dimensional images.

19. The method according to claim 18, further comprising
aligning, as a multiple two-dimensional images alignment step, tissue images between the plurality of two-dimensional images.
